(19) United States
(12) Patent Application Publication
(10) Pub. No.: US 2021/0163357 A1
(43) Pub. Date: Jun. 3, 2021

(54) Composition comprising at least one microorganism and use thereof

(71) Applicant: Evonik Operations GmbH, Essen (DE)

(72) Inventors: Tobias Müller, Roedermark (DE); Sarah Hintermayer, Duesseldorf (DE); Jan Hellriegel, Hanau (DE); Susanne Christine Martens-Kruck, Loerrach (DE); Isabelle Haas, Dortmund (DE); Lukas Falke, Bielefeld (DE); Stella Molck, Bielefeld (DE); Lorena Stannek-Göbel, Hannover (DE); Anke Reinschmidt, Essen (DE); Magnus Kloster, Rhede (DE)

(73) Assignee: Evonik Operations GmbH, Essen (DE)

(21) Appl. No.: 17/250,341

(22) PCT Filed: Jul. 8, 2019
(86) PCT No.: PCT/EP2019/068229

§ 371 Date: Jan. 7, 2021

(30) Foreign Application Priority Data
Jul. 11, 2018 (EP) 18182870.8

(51) Int. Cl. C04B 24/42; C04B 28/02; C12N 1/20; C12R 1/125

(52) U.S. Cl. CPC C04B 24/42; C04B 28/02; C04B 2103/0001; C12R 1/125; C12N 1/20

(57) ABSTRACT

A composition contains at least one microorganism which can form a phosphate or carbonate precipitate in an alkaline medium, and at least one calcium source. The composition has at least one silicon compound having at least one Si-atom, at least one C-atom, and at least one H-atom. The composition can be used in a method for producing a construction product.

The present invention relates to a composition comprising at least one microorganism capable of forming a phosphate or carbonate precipitate in an alkaline medium and optionally at least one calcium source, wherein the composition is characterized in that it comprises at least one silicon compound comprising at least one Si atom, at least one C atom and at least one H atom, and to a process for production of building products based on mineral building materials, wherein a corresponding composition is employed during production.

Built structures, in particular those based on mineral building materials, for example concrete built structures, are highly stressed as a result of environmental influences and/or strong mechanical stresses. This stress can result in cracks. Crack formation may, moreover, also be caused by structural influencing factors, such as for example, the storage of the component, or by climatic conditions which lead, for example, to the evaporation of water or to internal stresses as a result of temperature differences. Incipient cracks can allow water to penetrate into the built structures and cause lasting damage, inter ala through corrosion of steel reinforcements and through repeated freeze-thaw cycles. As a result concrete built structures have shortened lifetimes.

Numerous methods which attempt to prolong the lifetime of built structures based on mineral building materials, in particular of concrete built structures, are known.

These may be roughly distinguished into methods where additives are added to the building materials during construction of the built structures or production of the building materials and methods where the built structures/building materials are subsequently treated with additives. In this case, particular mention may be made of those processes in which cracks can be healed by the formation of expansive mineral structures or in which reaction resins or mineral systems under pressure are injected into the crack and thus subsequently seal the crack.

Known additives are for example hydrophobizing agents which during production of building materials, for example tiles, concrete parts, mortar or the like, are added thereto or after preparation of the building materials or built structures are applied thereto or to parts thereof.

Such hydrophobizing agents are described for example in EP 0538555 A1, WO 2006/081891 A1, WO 2006/081892 A1, WO 2013/076035 A1 or WO 2013/076036 A1.

While the use of hydrophobizing agents does prevent the water from penetrating into the concrete, if larger cracks occur due to stresses—penetration of water and weakening of the structure is not prevented.

Also known is the use of mineral-forming microorganisms as an additive in the production of building materials as described for example by Jonkers in WO 2009/093898 A1, WO 2011/126361 A1 and WO 2016/010434 A1 or as an additive for subsequent treatment of building materials/built structures as described for example by Jonkers in WO 2014/185781 A1, these being said to bring about self-healing of cracks.

Microorganisms can heal cracks up to a certain extent by formation of calcium carbonate, so-called MICB (microbial induced calcium precipitation), but if the amount of added Ca growth medium to be added is exhausted calcium carbonate (calcite) can no longer be produced either. However, the amount of Ca growth medium in the production of the building material is limited since above a certain amount of additive the density (concrete density) and thus the compressive strength markedly decreases. In a subsequent treatment of building materials/built structures said treatment must be repeated regularly to provide sufficient Ca growth medium. Further information may be found for example in Wiktor and Jonkers, Smart Mater. Struct. 25 (2016) “Bacteria-based concrete: from concept to market”, Qian et al., Front. Microbiol. 6:1225 (2015) “Self-healing of early age cracks in cement-based materials by mineralization of carbonic anhydrase microorganism.” and Lors et al. Construction and Building Materials 141:461-469 (2017) “Microbiologically induced calcium carbonate precipitation to repair microcracks remaining after autogenous healing of mortars”.

Combinations of hydrophobicity and microorganisms have also been previously described. Thus for example WO 2017/076635 A1 describes the production of hydrophobic, cement-containing compositions by addition of parts of biofilms produced by propagation of microorganisms on LB-agar sheets. Nano- or microscopic structures form on the surface and bring about the hydrophobicity. Prevention or reversal of cracks is not described here.

The problem addressed by the present invention was therefore that of providing a process that overcomes one or more of the disadvantages of the prior art solutions.

It was found that, surprisingly, this problem may be solved by compositions comprising at least one microorganism capable of forming a phosphate or carbonate precipitate in an alkaline medium and optionally at least one calcium source, wherein the composition is characterized in that it comprises at least one silicon compound comprising at least one Si atom, at least one C atom and at least one H atom.

The present invention accordingly provides compositions comprising at least one microorganism capable of forming a phosphate or carbonate precipitate in an alkaline medium and optionally at least one calcium source, wherein the compositions are characterized in that they comprise at least one silicon compound comprising at least one Si atom, at least one C atom and at least one H atom.

The present invention likewise provides a process for production of building products based on mineral building materials, wherein a composition according to the invention is employed during production.

The compositions according to the invention have the advantage that they may be employed both as a mass additive, for example in the production of building products or built structures, and as an additive/treatment for repair/maintenance of existing building products or built structures. In particular, repeated crack healing by the composition according to the invention is possible. The compositions according to the invention show, moreover, sufficient stability even without encapsulation of the biomass.

The combination of a silicon compound (as a hydrophobizing agent) comprising at least one Si atom, at least one C atom and at least one H atom and microorganisms additionally has the advantage that the hydrophobizing agents initially prevent penetration of water but if this barrier were to be broken the microorganisms can exert their healing effect (i.e. can at least partially seal the crack in the concrete by formation of preferably inorganic substances). The hydrophobizing properties displace water from the porous concrete structure for longer and this allows bacteria to remain in the sporulated state for longer or to resporulate more rapidly after previous activation.

A positive synergistic effect between hydrophobization and microorganisms is also observed: The strength of the concrete is improved compared to the strength of compositions containing only microorganisms and no hydrophobizing agents.

The use of the composition according to the invention already during production of concrete allows advancing damage of concrete built structures to be reduced in good time. This markedly extends the life cycles of concrete built structures while avoiding complex and, for bridge structures or high-rise structures, in some cases dangerous repair work. By the use of the composition according to the invention, built structures additionally need to be inspected for crack formation less often. The outlay for the inspection can thus be reduced. In addition, by the use of the composition according to the invention it is possible to avoid costs which may arise as a result of developed cracks remaining unnoticed and thus leading to progressive damage which then needs to be extensively rectified.

The reduced amount of concrete required as a result of the longer lifetime of concrete built structures likewise makes it possible to markedly reduce anthropogenic CO₂ production resulting from cement production.

The compositions according to the invention and the process according to the invention are exemplarily described below without any intention that the invention should be confined to these exemplary embodiments. Where ranges, general formulae or classes of compounds are specified hereinbelow, these are intended to encompass not only the corresponding ranges or groups of compounds which are explicitly mentioned but also all subranges and subgroups of compounds which can be obtained by leaving out individual values (ranges) or compounds. Where documents are cited in the context of the present description, their content shall fully form part of the disclosure content of the present invention, particularly in respect of the matters referred to. Where figures are given in percent hereinbelow, these are percentages by weight unless otherwise stated. Where averages, for example molar mass averages, are reported hereinbelow, these are the numerical average unless otherwise stated. Where properties of a material are referred to hereinbelow, for example viscosities or the like, these are properties of the material at 25° C. unless otherwise stated. When chemical (empirical) formulae are used in the present invention, the reported indices may be either absolute numbers or average values. The indices relating to polymeric compounds are preferably average values.

The composition according to the invention comprising at least one microorganism capable of forming a phosphate or carbonate precipitate in an alkaline medium and optionally at least one phosphate and/or calcium source has the feature that the composition comprises at least one silicon compound comprising at least one Si atom, at least one C atom and at least one H atom.

The microorganism is preferably selected from a bacterium, a lyophilized bacterium and a bacterial spore of the bacterium and is preferably a bacterial spore of a bacterium.

The microorganism is preferably selected from bacterial spores or bacteria of the genera Enterococcus, Diophrobacter, Lysinbacillus, Planococcus, Bacillus, Proteus or Sporosarcina, preferably selected from the bacterial spores or bacteria of the group comprising the species Bacillus cohnii, Bacillus megaterium, Bacillus pasteurii, Bacillus pseudofirmus, preferably Bacillus pseudofirmus (DSM 8715), Bacillus sphaericus, Bacillus spp., Bacillus subtilis, Proteus vulgaris, Bacillus licheniformis, Diophrobacter sp., Enterococcus faecalis, Lysinbacillus sphaericus, Proteus vulgaris and Sporosarcina pasteurii, particularly preferably Bacillus subtilis or Bacillus cohnii, very particularly preferably Bacillus subtilis, especially preferably Bacillus subtilis DSM 32315 as described in WO 2017/207372 A1 and as deposited at the DSMZ, Inhoffenstraße 7B, 38124 Braunschweig, Germany on 16 Dec. 2015 under the regulations of the Budapest Treaty on the International Recognition of the Deposit of Microorganisms for the Purposes of Patent Procedure under the abovementioned number and in the name of Evonik Degussa GmbH or mutants thereof having all identifying characteristics of the strain DSM 32315 and preferably having a DNA sequence identity to strain DSM 32315 of at least 95%, preferably at least 96, 97 or 98%, particularly preferably at least 99% and yet more preferably 99.5%, or Bacillus subtilis (DSM 10). It is very particularly preferred when the microorganism is a bacterial spore of the recited preferred bacteria.

It may be advantageous when the weight ratio of microorganisms capable of forming a phosphate or carbonate precipitate in an alkaline medium to silicon compounds comprising at least one Si atom, at least one C atom and at least one H atom in the composition is from 100:1 to 1:100, preferably from 10:1 to 1:2.

It is preferable when the mass fraction of microorganisms capable of forming a phosphate or carbonate precipitate in an alkaline medium based on the total mass of the composition, preferably on the total mass of the composition without accounting for water, is from 0.0001% to 10% by weight, preferably from 0.001% to 5% by weight and particularly preferably from 0.002% to 3% by weight.

If the employed microorganisms are employed as spores the number of spores per gram is preferably from 1×10⁵ to 1×10¹³ spores/g, preferably from 1×10⁷ to 1×10¹² spores/g and particularly preferably from 1×10⁹ to 1×10¹¹ spores/g. The spore number may be determined according to the standard DIN EN 15784.

The composition according to the invention preferably contains at least one mineral building material, preferably cement. The composition according to the invention may also comprise a plurality of mineral building materials. The composition according to the invention may in principle contain any known mineral building materials. The composition may preferably contain as mineral building materials sand, clay, gravel, crushed stone and/or gypsum, particularly preferably in combination with cement.

The composition according to the invention may contain a solvent, i.e. constitute a liquid-containing mixture or may be solvent-free, i.e. constitute a dry mixture. Preferred compositions according to the invention are those which contain a solvent, in particular water.

If the composition according to the invention contains a solvent, in particular water, the proportion of solvent, preferably water, in the total composition is from 2.5% to 66% by weight, preferably from 5% to 40% by weight and particularly preferably from 10% to 20% by weight.

It may be advantageous when the composition according to the invention contains an enrichment medium (often also called a growth medium or substrate) for enrichment of the microorganisms. Enrichment media that may be used include any known enrichment media. The enrichment medium preferably comprises a carbon source and/or a nitrogen source and the enrichment medium particularly preferably also contains a phosphorus source, in particular a phosphate source. Preferred carbon sources are selected from the group of monosaccharides, oligosaccharides and polysaccharides. Particularly preferred carbon sources are glucose, fructose, maltose, saccharose, molasses, starch and starch products as well as whey and whey products. The starch and starch products are preferably obtained from wheat or maize. Also employable as a carbon source are alditols (sugar alcohols) including in particular glycerol. Suitable nitrogen sources include both organic and inorganic nitrogen sources. Organic nitrogen sources are preferably selected from the group consisting of peptone, yeast extract, soy flour, soy husk, cottonseed flour, lentil flour, aspartate, glutamate and triptic soy broth. A preferred inorganic nitrogen source is ammonium sulfate. Some of the recited carbon sources are also suitable as a nitrogen source and vice versa, these include for example whey and whey products, peptone, yeast extract, soy flour, soy husk, cottonseed flour, lentil flour, triptic soy broth. The phosphorus source/phosphate source is preferably selected from the group consisting of ammonium phosphate, sodium phosphate and potassium phosphate. Phosphorus may also be a constituent of the carbon and/or nitrogen sources. The composition of the enrichment medium based on the respective dry weights of the individual components is dependent on the respective nutrient spectrum but the weight ratio is preferably 1:0.01:0.001 to 1:10:10 for carbon source:nitrogen source:phosphorus source (C:N:P components). Suitable enrichment media are described for example in: “FAO. 2016. Probiotics in animal nutrition—Production, impact and regulation by Yadav S. Bajagai, Athol V. Klieve, Peter J. Dart and Wayne L. Bryden. Editor Harinder P. S. Makkar. FAO Animal Production and Health Paper No. 179. Rome.” (ISBN 978-92-5-109333-7). The composition according to the invention preferably contains a tryptic soy broth, a yeast extract, a peptone, an aspartate or a glutamate or a mixture of two or more of the recited enrichment media. The composition according to the invention particularly preferably contains tryptic soy broth (casein-soy-peptone medium) as an enrichment medium. It may be advantageous when the enrichment media contain not only the recited agents but also one or more trace elements. The composition according to the invention preferably contains enrichment medium in an amount such that the mass ratio of enrichment medium to microorganisms in the composition is from 10,000:1 to 1:10,000, preferably from 1000:1 to 1:1000, more preferably from 100:1 to 1:100, particularly preferably from 10:1 to 1:10.

It may be advantageous when the composition according to the invention comprises a calcium source. As the calcium source the composition according to the invention by preference comprises calcium salts, preferably calcium salts of organic acids. Particularly preferred calcium sources are those which can simultaneously also function as an enrichment medium. Particularly preferred calcium sources are calcium gluconate, calcium acetate, calcium formate, calcium lactate or calcium nitrate, very particularly preferably calcium lactate.

The at least one silicon compound which contains at least one Si atom, at least one C atom and at least one H atom is preferably selected from silane compounds, siloxane compounds, silicone oils, siliconates, organosilane compounds or organosiloxane compounds, preferably selected from organosilane compounds.

The at least one silicon compound preferably has hydrophobizing properties. Particularly preferred silicon compounds having hydrophobizing properties are those which reduce the water absorption of mortar determined according to DIN EN 480-5 by at least 50% after 7 days and by at least 60% after 28 days when these are admixed with the mortar in a concentration of 5% by weight, preferably in a concentration of 2% by weight and particularly preferably in a concentration of 0.5% by weight based on the cement.

The composition according to the invention preferably contains at least one silicon compound which comprises at least one Si atom, at least one C atom and at least one H atom and conforms to the formula (I), (IIa) or (IIb),

R—SiR¹ₓR²_{z} (I)

in which
R is a linear or branched alkyl group having 1 to 20 C atoms,
R¹ is a linear or branched alkyl group having 1 to 4 C atoms,
R² is a linear or branched alkoxy group having 1 to 4 C atoms or a hydroxyl group, wherein the radicals R¹ and R² may each be identical or different,
x equals 0, 1 or 2,
z equals 1, 2 or 3 and x+z=3,

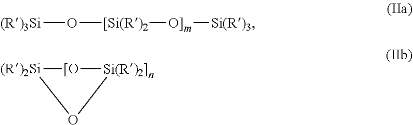

in which the individual radicals R′ independently of one another represent hydroxyl, alkoxy, by preference having 1 to 6, preferably having 1 to 4, carbon atoms, alkoxyalkoxy, by preference having 1 to 6, preferably having 1 to 4, carbon atoms, alkyl, by preference having 1 to 20, preferably having 1 to 10, carbon atoms, alkenyl, by preference having 1 to 20, preferably having 1 to 10, carbon atoms, cycloalkyl, by preference having 1 to 20, preferably having 1 to 10, carbon atoms and/or aryl, by preference having 1 to 20,
preferably having 1 to 10, carbon atoms,
m is an integer from 2 to 30,
n is an integer from 3 to 30,
with the proviso that sufficient of the radicals R′ in the compounds of formulae (IIa) and (IIb) are an alkoxy radical to ensure that the quotient of the molar ratio of Si to alkoxy radicals in the compounds of formulae (IIa) and (IIb) is at least 0.3, in particular at least 0.5. The composition may also contain mixtures of compounds of formulae (I), (IIa) and/or (IIb).

The formula (IIb)

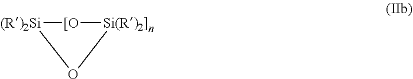

is in this case equivalent to the formula

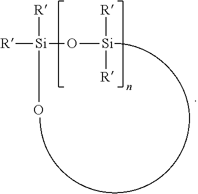

The composition according to the invention preferably contains at least one silicon compound which comprises at least one Si atom, at least one C atom and at least one H atom and is selected from CH₃Si(OCH₃)₃, CH₃Si(OC₂H₅)₃, C₂HSi(OCH₃)₃, i-C₃H₇Si(OCH₃)₃, C₂H₅Si(OC₂H₅)₃, i-C₃H₇Si(OC₂H₅)₃, n-C₃H₇Si(OCH₃)₃, n-C₃H₇Si(OC₂H₅)₃, i-C₃H₇Si(OCH₃)₃, n-C₄H₉Si(OCH₃)₃, n-C₄H₉Si(OC₂H₅)₃, i-C₄H₉Si(OCH₃)₃, n-C₄H₉Si(OC₂H₅)₃, n-C₅H₁₁Si(OCH₃)₃, n-C₅H₁₁Si(OC₂H₅)₃, i-C₅H₁₁Si(OCH₃)₃, i-C₅H₁₁Si(OC₂H₅)₃, n-C₆H₁₃Si(OCH₃)₃, n-C₆H₁₃Si(OC₂H₅)₃, i-C₈H₁₃Si(OCH₃)₃, i-C₆H₁₃Si(OC₂H₅)₃, n-C₈H₁₇Si(OCH₃)₃, n-C₈H₁₇Si(OC₂H₅)₃, i-C₈H₁₇Si(OCH₃)₃, i-C₈H₁₇Si(OC₂H₅)₃, n-C₁₀H₂₁Si(OCH₃)₃, n-C₁₀H₂₁Si(OC₂H₅)₃, i-C₁₀H₂₁Si(OCH₃)₃, i-C₁₀H₂₁Si(OC₂H₅)₃, n-C₁₆H₃₃Si(OCH₃)₃, n-C₁₆H₃₃Si(OC₂H₅)₃, i-C₁₆H₃₃Si(OCH₃)₃, i-C₁₆H₃₃Si(OC₂H₅)₃ or partial condensates of one or more of the recited compounds or a mixture of the recited compounds, a mixture of the partial condensates or a mixture of the compounds and the partial condensates.

Compounds according to formula (IIa) or (IIb) may be for example methylalkoxysiloxanes, ethylalkyoxysiloxanes, propylalkoxysiloxanes, butylalkoxysiloxanes, hexylalkoxysiloxanes, phenylalkoxysiloxanes, octylalkyoxysiloxanes or hexadecylalkoxylsiloxanes, wherein alkoxy by preference represents methoxy or ethoxy, preferably methoxy.

It may be advantageous when the composition according to the invention comprises further additives. Particularly when it comprises one or more mineral building materials, preferably cement, particularly preferably cement and sand or gravel, the composition according to the invention preferably comprises further concrete or mortar additives, in particular selected from shrinkage reducers, defoamers, (super) plasticizers, accelerants, retardants, air entrainment agents, rheology modifiers, fillers/intergrinding materials and/or fibres. The mass fraction of all further additives in the total composition is by preference from 0% to 40% by weight, preferably 0.5% to 25% by weight and particularly preferably 1% to 10% by weight.

As (super) plasticizers the compositions according to the invention preferably comprise polycarboxylate ethers, lignosulfonates, melamine sulfonates, casein or polynaphthalene sulfonates or mixtures of two or more of the recited compounds. If the composition according to the invention contains (super) plasticizers the proportion thereof in the composition according to the invention is preferably from 0.01% to 2% by weight, preferably from 0.05% to 0.5% by weight.

As shrinkage reducers the compositions according to the invention by preference comprise monoalcohols, glycols, preferably neopentyl glycol, alkanediols, polyoxyalkylene glycols, aminoalcohols or polyoxyalkylenes or mixtures of two or more of the recited compounds.

As defoamers the compositions according to the invention preferably comprise mineral oil, polyethers, acetylene compounds or vegetable oils or mixtures of two or more of the recited compounds.

As accelerants the compositions according to the invention preferably comprise CaCl₂, carbonates, preferably Na₂CO₃ or Li₂CO₃, aluminates, preferably tricalcium aluminate, CaO or sulfates or mixtures of two or more of the recited compounds. If the compositions according to the invention comprise Ca-containing substances as accelerants, addition of calcium sources may optionally be eschewed.

As retarders the compositions according to the invention by preference comprise carbohydrates, preferably monosaccharides, disaccharides, oligosaccharides and/or polysaccharides, lignin sulfonates, hydroxycarboxylic acids, phosphates, tetraborates, citric acid, tartaric acid, tartrates or citrates or mixtures of two or more of the recited compounds. Some of the retarders may optionally also be suitable as an enrichment medium. If such retarders are employed the proportion thereof is counted as part of the mass fraction of enrichment medium.

As air entrainment agents the compositions according to the invention by preference comprise betaine, natural resins, preferably root resin or tall oil rosin, lauryl sulfate, sulfosuccinates, fatty acids, sulfonates, soaps or fatty (acid) soaps or mixtures of two or more of the recited compounds. Some of the air entrainment agents such as for example sulfosuccinates and fatty acids may optionally also be suitable as enrichment medium. If such air entrainment agents are employed the proportion thereof is counted as part of the mass fraction of enrichment medium.

If the composition according to the invention contains shrinkage reducers, defoamers, accelerators, retarders and/or air entrainment agents the sum of the proportions thereof in the composition according to the invention is preferably from 0.01% to 10% by weight, preferably from 0.02% to 3% by weight and particularly preferably from 0.05% to 0.5% by weight.

As rheology modifiers the compositions according to the invention preferably comprise starch, cellulose ethers, PVAL, guar gum, xanthan gum, welan gum, alginates, agar, polyethylene oxides, bentonite or polyacrylamide or mixtures of two or more of the recited compounds. Some of the rheology modifiers such as for example starch and cellulose may optionally also be suitable as enrichment medium. If such rheology modifiers are employed the proportion thereof is counted as part of the mass fraction of enrichment medium.

As fillers/intergrinding materials the compositions according to the invention preferably comprise fly ash, limestone flour, blast furnace slag, rock flours, micro- or nanosilica or mixtures of two or more of the recited compounds.

As fibres the compositions according to the invention preferably comprise steel fibres, plastics fibres (PAN), glass fibres or carbon fibres or mixtures of two or more of the recited fibres.

Particularly if they comprise no solvent the compositions according to the invention may also comprise carrier materials, such as are described for example in Wiktor and Jonkers, Smart Mater. Struct. 25 (2016) “Bacteria-based concrete: from concept to market”.

The compositions according to the invention may be used for production of building products or built structures. It is preferable when the compositions according to the invention are used in the process for production of building products described hereinbelow.

The process according to the invention for production of building products, preferably based on mineral building materials, has the feature that at least one of the abovementioned compositions according to the invention is employed during production.

The building product to be produced with the process according to the invention is preferably mortar, mortar-based components/products, steel-reinforced concrete, concrete, a (steel-reinforced) concrete part, a concrete block, a roof tile, a brick or a porous concrete block.

In the process according to the invention the composition according to the invention may be employed before or after completion of the building product or of the built structure. The composition according to the invention is preferably employed before completion of the building product or of the built structure.

If the composition according to the invention is employed before completion of the building product or of the built structure the addition is preferably carried out in a mixing process, particularly preferably during a mixing process, that must also be used in the production of the building product from conventional components.

If the composition according to the invention is employed after completion of the building product or of the built structure the application of the composition is preferably effected by application of the composition onto the surface of the building product or of the built structure. Application may be effected by spray application or brush application of the composition onto building products or built structures and in the case of smaller building products such as for example tiles or premade concrete parts application by immersion of the building products in the composition may also be suitable. The composition may be employed free from cement, by preference as a liquid composition, preferably as a sprayable composition, or as a cement-comprising composition, for example in the form of mortar. Compositions according to the invention which are free from cement are preferably used for surface treatment of building products or built structures exhibiting small cracks, preferably cracks having a crack width of less than 1 mm. In the case of larger cracks it is preferable to employ a composition comprising cement.

The phosphate or carbonate precipitate, in particular calcium carbonate, formed by the composition according to the invention can partially or fully fill or close pores, contact surfaces, joints, cracks, fractures or cavities in or on a component. The composition according to the invention is suitable as a mass additive for use in concrete, prefabricated concrete parts, concrete blocks or fiber concrete sheets or else as a mass additive for use in other mineral building materials which depending on the composition of the mass additive allow formation of phosphate or carbonate precipitates, in particular calcium carbonate, or other mineral structures. The composition according to the invention is also suitable for subsequent treatment of concrete, prefabricated concrete parts, concrete blocks, fiber concrete sheets or built structures. Said composition may be applied subsequently by spraying or brushing for example. It is also possible for only individual constituents of the composition, such as for example a nutrient solution or other auxiliary substances for activating the already present microorganisms, to be applied subsequently.

In a preferred embodiment the composition according to the invention further results in a specific metabolization of other additives (preferably of additives which after curing are present in the component without further function, for example concrete flow agents) or of other specifically introduced substances (in order for example to generate a specific pore structure) or of penetrating substances with potential to damage the component (for example substances aggressive towards concrete).

Also preferred is the use of the composition according to the invention for coating or combined use with installed components, shoring or sealing elements in the concrete, mortar or other, preferably cementitious, building materials, for example in conjunction with sealing sheets (for example incorporated in a coating or in a nonwoven fabric) to prevent water penetration behind the sheet through the formation of mineral structures, in conjunction with a sealing sheet to bring about a specific “coalescence” of the sealing layer and the component, in conjunction with joint seals to prevent potential water penetration around the joint through the formation of mineral structures or in conjunction with other installed components to produce a watertight join. The installed components are preferably selected from spacers, formwork anchors, pipe feedthroughs or other feedthroughs.

Also preferred is the use of the composition according to the invention in conjunction with metallic but preferably nonmetallic shoring and reinforcing elements. Particularly in the case of nonmetallic shoring and reinforcing elements insufficient adhesive bonding and thus potential penetration of water behind the elements or only limited force transfer may occur. The composition according to the invention makes it possible to achieve sufficient adhesive bonding, thus reducing penetration of water behind the elements, and to improve force transfer. Nonmetallic shoring and reinforcing elements are for example polymeric shoring elements, such as fiber-reinforced epoxy resin systems or glass or carbon fibers. The composition according to the invention may also be a constituent of the fiber size.

The composition according to the invention is suitable preferably for filling or sealing cracks in multilayered systems, for example in tunnel construction or in triple walls in the construction of prefabricated concrete components. The composition is preferably used for filling cavities, pores, capillaries or joints resulting from processing. The composition according to the invention may also be used in conjunction with modular components (blocks, prefabricated components, plugs) to allow a specific “coalescence” to afford a joined component.

The composition according to the invention may additionally be used as a constituent of a coating or sealing system (for example mineral sealing slurries etc.), as a constituent of an injection system for fracture, joint, floor, aggregate or cavity injection, as a constituent of an aftertreatment composition (to allow for rapid sealing of a superficial pore structure and thus reduce evaporation of water) as a constituent of a joint mortar in order to prevent moisture rising by capillary action for example or as a constituent of an adhesive system for specific “coalescence” of components.

The composition according to the invention may be liquid or solid. In solid form it is preferably in particulate form, in particular as a powder or granulate. This makes the composition more readily handleable, in particular more readily pourable and easier to meter. The particles, in particular the powder or granulate may be encapsulated or coated. A suitable encapsulation/coating agent is in particular polyvinyl alcohol. The composition is preferably in unencapsulated/uncoated form.

The composition according to the invention may be employed as a one-, two- or multi-component system. As a two- or multi-component system the two or more components are stored separately and mixed with one another only shortly before or during use.

The composition according to the invention is employed preferably in built structures such as for example sewage works and channels, residential and administrative buildings (preferably basements), infrastructure (for example bridges, tunnels, troughs, concrete roads, parking garages and multistorey carparks), hydraulic structures (for example locks and harbor installations), energy sector built structures (for example wind turbines, cooling towers, biogas plants, pumped storage plants). Particular preference is given in particular to the use of the composition according to the invention in components in contact with the earth or exposed to weathering, such as for example exterior walls or foundations.

Even without further elaboration it is assumed that a person skilled in the art will be able to utilize the description above to the greatest possible extent. The preferred embodiments and examples are therefore to be interpreted merely as a descriptive disclosure which is by no means limiting in any way whatsoever.

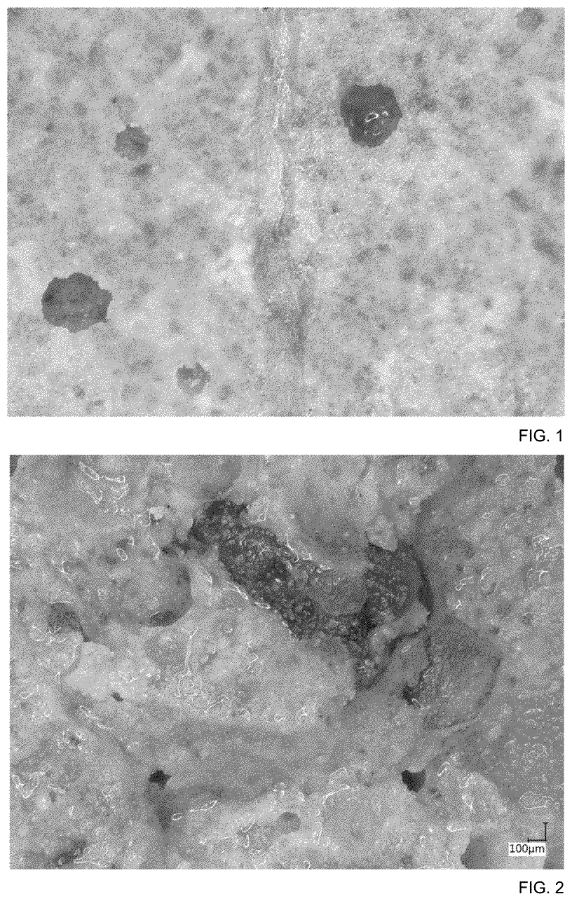

The subject-matter of the present invention is more particularly elucidated with reference to FIGS. 1 to 4, without any intention that the subject-matter of the present invention be restricted thereto.

The image in FIG. 1 shows a 200-times magnification of the side view of the test specimen comprising a crack, 18 days after the block from example 4a was broken in two. It is apparent that the crack has been healed.

The image in FIG. 2 shows a 100-times magnification of a top-down view onto the fracture surface, 69 days after the block from example 4a was broken in two. It is apparent that Ca carbonate has formed in the crack.

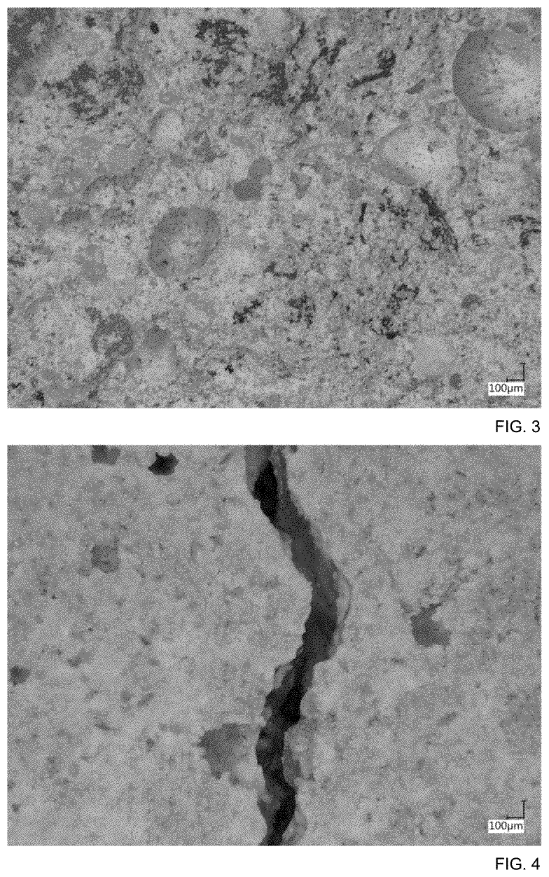

The image in FIG. 3 shows a 100-times magnification of a top-down view onto the fracture surface, 0 days after the block from example 4b was broken in two. It is apparent that healing has not yet occurred.

The image in FIG. 4 shows a 100-times magnification of the side view of the test specimen from example 4c, 69 days after the block from example 4c was broken in two. It is apparent that Ca carbonate has formed in the crack.

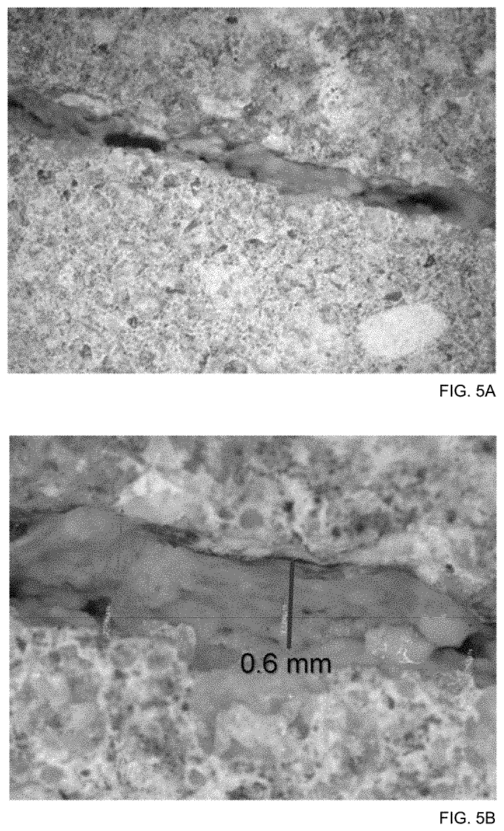

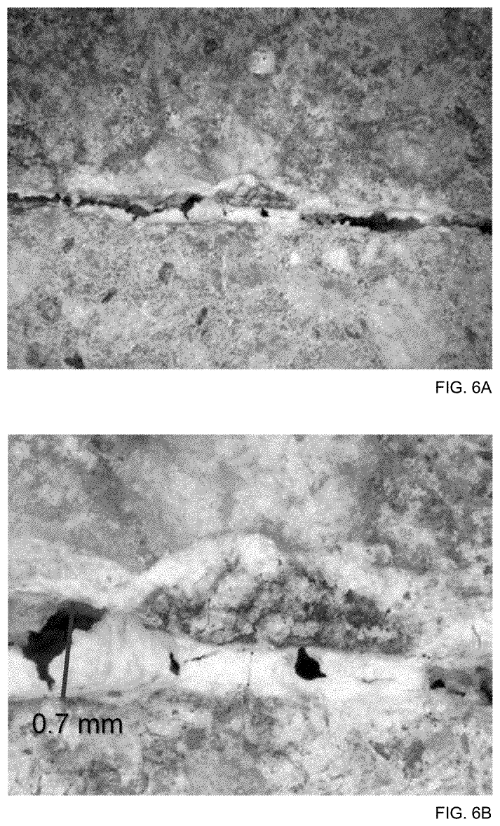

The images in FIGS. 5a and 5b show in 30- and 100-times magnification the crack in the test specimen of example 3 (E). The images in FIGS. 6a and 6b show in 30- and 100-times magnification the crack in the test specimen of example 3 (S). In both cracks the formation of filling material (crack healing) is readily apparent after one day.

The subject-matter of the present invention is elucidated in detail in the examples which follow, without any intention that the subject-matter of the present invention be restricted to these.

### Measurement Methods:

The healing of the cracks was determined optically using a microscope.
Flexural tensile strengths were determined based on DIN EN 12390-5 (3-point flexural test with central loading).
Karsten tube test: Water absorption was measured using a water penetration tester, also known as a Karsten tube as described in “MEASUREMENT OF WATER ABSORPTION UNDER LOW PRESSURE; RILEM TEST METHOD NO. 11.4, horizontal application” (https://www.m-testco.com/files/pages/Rilem%20Test.pdf)

### Substances Used:

Spores of Bacillus subtilis (DSM 32315), also referred to hereinbelow as spores 32315, 8×10¹⁰ spores/g (spore number determined according to the standard DIN EN 15784).
Spores of Bacillus subtilis (DSM 10)
Spores of Bacillus pseudofirmus (DSM 8715)
Tryptic Soy Broth (Sigma Aldrich, product number 22092), also referred to hereinbelow as TSB
Milke® Classic CEM I 52.5 N cement (Heidelberg Cement AG), also referred to hereinbelow as cement
CEN standard sand according to DIN EN 196-1 (Normensand GmbH), also referred to hereinbelow as standard sand,
Liquid Repair System-ER7 (Basilisk-Contracting BV), also referred to hereinbelow as LRS
Protectosil® WS 405 (Evonik Resource Efficiency GmbH), an aqueous silane emulsion also referred to hereinbelow as WS 405
Protectosil® WA CIT (Evonik Resource Efficiency GmbH), an aqueous emulsion of multifunctional silanes also referred to hereinbelow as WA CIT
Meat extract (Merck KGaA)
Peptone from casein (Merck KGaA)
Concrete cubes, sawn similarly to ISO 13640, method 1 concrete quality according to EN 196 CEM I 42.5, edge length 5 cm, from Rocholl GmbH
Kuraray Poval® 4-88 (Kuraray), polyvinyl alcohol
Kuraray Elvanol® 8018 (Kuraray), polyvinyl alcohol copolymer with lactone

### EXAMPLES

### Example 1: Testing of Compatibility of Microorganisms with Hydrophobizing Agent and Shrinkage Reducer

The strains Bacillus subtilis (DSM 10) and Bacillus pseudofirmus (DSM 8715) were investigated for compatibility with hydrophobizing agents and shrinkage reducers.

A mixture of 3 g of meat extract, 5 g of peptone from casein and 1000 mL of distilled water adjusted to pH 7 using HCl/NaOH for Bacillus subtilis (DSM 10) and adjusted to pH 7 using Na sesquicarbonate for Bacillus pseudofirmus (DSM 8715) was used as the medium.

A pre-culture was initially produced for each of the two strains: To this end, an inoculation dose of the spores was in each case placed into a culture tube with 8 mL of the respective medium and left overnight in a laboratory shaker at 30° C. and 200 revolutions per minute. Furthermore, aqueous stock solutions respectively having a concentration of Protectosil® WS405 (hydrophobizing agent) of 500 g/L and a concentration of neopentyl glycol (shrinkage reducer) of 280 g/L were produced.

For the main cultures two 6-well spot plates were each filled with 8 mL of medium. Then, 10 μL of the first pre-culture were added to each well of the first plate and 10 μL of the second pre-culture were added to each well of the second plate. Aqueous PROTECTOSIL® WS405 stock solution was added to three wells of both plates in amounts such that the concentration of PROTECTOSIL® WS405 was 5 g/L, 20 g/L or 30 g/L. Neopentyl glycol stock solution was added to the other three wells of the two plates in amounts such that the concentration of neopentyl glycol was 0.7 g/L, 7 g/L or 14 g/L.

The main cultures were subsequently left in a laboratory shaker for 4 days at 30° C. and 200 revolutions per minute. Observation of turbidity changes were used to determine whether the microorganisms grow in the presence of hydrophobizing agent and/or shrinkage reducer.

It was found that the growth of neither organism was impaired by the addition of hydrophobizing agent or shrinkage reducer in the recited concentrations.

For spores of the strain Bacillus subtilis DSM 32315 compatibility with neopentyl glycol (7 g/L) and Protectosil® WS405 (20 g/L) was investigated on agar plates. A mixture of 3 g of meat extract, 5 g of peptone from casein and 1000 mL of distilled water adjusted to pH 7 using HCl/NaOH was used as medium. Formation of colonies was observed in all cases. This shows that the additives do not influence the growth of the strain.

### Example 2: Production of Test Specimens

Test specimens were produced using the formulation for producing standard mortar having a mortar composition according to EN 480-1. To this end, 450 g of Milke® classic CEM I 52.5 N cement and 1350 g of CEN standard sand according to EN 196-1 were homogenized to afford a dry mixture using a mortar mixer from Hobart.

The homogenized dry mixture was added to the mortar mixer over 30 seconds at a slow mixing speed (setting 1). 450 g of water were then added over 30 seconds and the total mortar mixture was stirred for a further 60 seconds at the slow setting. The amount of water was chosen such that the weight ratio of water to cement was 1 to 2.

The mortar was then stirred for 60 seconds at high speed (setting 2). The total mixing time ran to 3 minutes and 30 seconds.

Steel moulds for three prisms in each case (4 cm×4 cm×16 cm) were filled to an overfill of 0.5 to 1.0 cm using a box attachment and subsequently compacted on a vibration table for 120 seconds at 50 Hz. The mortar in the mould was then smoothed and covered with a glass sheet. After 48 hours the prisms were carefully demoulded, labelled and stored under standard climatic conditions until testing after 28 days.

### Example 3: Testing of the Healing Effect of Compositions

A number of test specimens from example 2 were broken apart in the middle and treated at the fracture edges either with a prior art composition (S) or with an inventive composition (E) which, however, lacked hydrophobizing agent and subsequently joined together again.

The treatment with the Liquid Repair System-ER7 (prior art product) is carried out such that 90 g of the component A in 500 mL of water (temperature of the water 40° C.) was converted into solution A and 50 g of component B in 250 mL of water (temperature of the water 40° C.) was converted into solution B in accordance with the use instructions. Then, according to the use instructions, the fracture edges were sprayed twice with solution A and then once with solution B.

The treatment with the composition according to the invention was carried out such that initially 15 g of tryptic soy broth were stirred with 50 g of spores of Bacillus subtilis DSM 32315 in 500 mL of water and this solution was sprayed onto the fracture edges.

After joining the test specimens were secured with a Teflon tape. The test specimens were stored in a water bath at room temperature. The test specimens were immersed into the water bath to a depth of 0.5 cm and the crack was not below the water level. The crack was sprayed with water at regular intervals of 2 days.

Crack healing was observed for both test specimens (FIGS. 5a, 5b, 6a and 6b) even after a time of 1 day. It was possible to exert a vertical downward force of at least 1.23 N on the healed crack in each case. This force corresponds to the mass of the lower part of the test specimen multiplied by the acceleration due to gravity of 9.81 m/s². As a reference one test specimen was brush coated with exclusively tryptic soy broth. No healing of the crack was observable here after the same time had elapsed.

### Example 4: Production of Test Specimens with Addition of Healing Additives

The production of the test specimens was performed as described in example 2. However, the aqueous proportion of the added compositions was considered as forming part of the mixing water and thus accounted for and all mixtures for producing test specimens were therefore produced with the same water to cement ratio to ensure comparability of results. The employed substances and the appearance of the mortar mixtures during processing are reported in table 1a.

TABLE 1a
Mortar mixtures (without water fraction) and appearance thereof

| | | Standard | | 32315 | | | | |
|---|---|---|---|---|---|---|---|---|
| Example | Cement | sand | LSR | spores | TSB | WS 405 | WA CIT | Appearance |
| 4a | 450 g | 1350 g | | 13.5 g | 4.5 g | — | — | normal viscosity |
| 4b | 450 g | 1350 g | | 13.5 g | 4.5 g | 18 g | | normal viscosity |
| 4c | 450 g | 1350 g | | 13.5 g | 4.5 g | | 18 g | normal viscosity |

To assess the hydrophobizing effect of the silane addition (silicon compound comprising at least one Si atom, at least one C atom and at least one H atom) the reduction in capillary water absorption over a period of 24 h and 14 days was determined. Test specimen 4a (only biomass) was used as a reference.

Before commencement of water absorption the dry mass of each test specimen was determined. Each test specimen was then stored vertically with the 40 mm×40 mm base surface in a constant water depth of 3 mm in a suitable container. Suitable blocks or linings (glass inserts or glass beads) are to be used to ensure unhindered access of the water to the immersed base surface. The individual test specimens must not contact one another and the container is to be closed for the duration of the test. The masses of the individual test specimens are to be determined and noted in the test protocol after the specified time intervals. In order to remove adherent water at the test specimens are lightly dabbed with a dry cloth (test setup analogous to EN 480-5 but with other measurement periods and without triplicate determination). The percentage reduction in water absorption was determined by the following method:

$$100 - \frac{\begin{matrix}[ ( mass ( ex ) after UWS - mass ( ex ) before UWS ) / \\ mass ( ex ) before UWS * 100 ]\end{matrix}}{\begin{matrix}[ ( mass ( ref ) after UWS - mass ( ref ) before UWS ) / \\ mass ( ref ) before UWS * 100 ]\end{matrix}} * 100$$

ref=reference example (4a); ex=examples (4b/4c)

The results after 24 hours are shown in table 1b, the results after 14 days are shown in table 1c.

TABLE 1b
reduction in capillary water absorption after 24 h

| | | | Water | Reduction |
|---|---|---|---|---|
| | Mass before | Mass after 24 h | absorption | in WA after |
| Example | UWS [g] | UWS [g] | [g] | 24 h [%] |
| 4a | 518.3 | 555.1 | 36.8 | — |
| 4b | 531.3 | 534.6 | 3.3 | 91.3 |
| 4c | 535.3 | 540 | 4.7 | 87.6 |

UWS—underwater storage;
WA—water absorption

TABLE 1c
reduction in capillary water absorption after 14 d

| | | | Water | Reduction |
|---|---|---|---|---|
| | Mass before | Mass after 14 d | absorption | in WA after |
| Example | UWS [g] | UWS [g] | [g] | 14 d [%] |
| 4a | 518.3 | 557.0 | 38.7 | — |
| 4b | 531.3 | 539.4 | 8.1 | 79.7 |
| 4c | 535.3 | 546.5 | 11.2 | 72.1 |

UWS—underwater storage;
WA—water absorption

As is apparent from tables 1b and 1c addition of a silicon compound comprising at least one Si atom, at least one C atom and at least one H atom (of a hydrophobizing agent) markedly reduces the water absorption of the test specimens.

The test specimens were then broken into two parts, placed on top of one another again at the fracture edges and subsequently stored standing upright in a bowl of water (about 5 mm water fill height) for 69 days so that the fracture was immersed in the water on one side.

In 200-times magnification a side view of the test specimen comprising the crack showed that 18 days after the block from example 4a was broken in two the crack was healed (filled) (FIG. 1). In 100-times magnification a top-down view onto the fracture surface showed that 69 days after the block from example 4a was broken in two Ca carbonate had formed in the crack (FIG. 2). The 100-times magnification of the side view of the test specimen from example 4c showed that 69 days after the block from example 4a was broken in two Ca carbonate had formed in the crack.

### Example 5: Influence of Microorganism Concentration and Ca Source

The aim was to determine the influence of the mass of microorganisms and additional Ca source on flexural strength and water absorption of the test specimen. To this end, test specimens with different combination options of biomass, tryptic soy broth, Ca source and hydrophobizing agent (WS405) were employed.

The production of the test specimens was performed as described in example 2. However, the components and concentrations reported in table 2a were used. The Ca source employed was calcium lactate hydrate. Example Si is the reference sample.

For simpler metering of the microorganisms 0.68 g of 32315 spores were initially diluted with 50 mL of tap water to produce a spore mixture which accordingly had a concentration of 0.0136 g (spores 32315)/mL.

TABLE 2a
Employed formulations for producing the test specimens

| | | Standard | | Spore | | | |
|---|---|---|---|---|---|---|---|
| Example | Cement | sand | Water | solution | TSB | Ca source | WS405 |
| 5a | 450 g | 1350 g | 224.0 g | 1 mL | 4.5 g | 0 g | 0 g |
| 5b | 450 g | 1350 g | 222.9 g | 1 mL | 4.5 g | 0 g | 2.25 g |
| 5c | 450 g | 1350 g | 224.0 g | 1 mL | 4.5 g | 3.15 g | 0 g |
| 5d | 450 g | 1350 g | 222.9 g | 1 mL | 4.5 g | 3.15 g | 2.25 g |
| 5e | 450 g | 1350 g | 224.9 g | 0.1 mL | 4.5 g | 0 g | 0 g |
| 5f | 450 g | 1350 g | 223.8 g | 0.1 mL | 4.5 g | 0 g | 2.25 g |
| 5g | 450 g | 1350 g | 224.9 g | 0.1 mL | 4.5 g | 3.15 g | 0 g |
| 5h | 450 g | 1350 g | 223.8 g | 0.1 mL | 4.5 g | 3.15 g | 2.25 g |
| 5i | 450 g | 1350 g | 225.0 g | 0 mL | 0 g | 0 g | 0 g |

After 28 days of storage of the test specimens at 23° C. and 50% relative humidity (standard climatic conditions) the flexural tensile strength of the test specimens and the reduction in the water absorption after 24 h were measured. To determine water absorption after 24 h the test specimens were stored standing upright in a water bath. They were immersed into the water to a depth of about 5 cm. After 24 h the amount of water absorbed by the test specimens was determined by gravimetric means. The results are shown in Table 2b.

TABLE 2b
Results of testing

| Example | S_{fracture} | Reduction in water absorption | Rating |
|---|---|---|---|
| 5a | 836.6N | −22.5% | − |
| 5b | 3016.3N | 65.3% | ++ |
| 5c | 294.6N | −36.4% | −− |
| 5d | 2547.2N | 65.7% | + |
| 5e | 565.9N | −40.8% | −− |
| 5f | 2808.6N | 68.2% | ++ |
| 5g | 727.3N | 6.8% | − |
| 5h | 2553N | 69.1% | + |
| 5i | 3849.4 N | 0.0% | |

It is apparent from the results shown in table 2b that markedly higher strengths are achievable with addition of TSB, microorganisms and hydrophobizing agent than without the addition of hydrophobizing agent. In addition, compared to the untreated test specimen water absorption increases (negative % values) without addition of hydrophobizing agent but with addition of TSB and spore solution. The further addition of a Ca source appears to result in a slight improvement in the reduction in water absorption but also to a slightly lower flexural strength.

### Example 6: Effect of Surface Treatment

The aim of this experiment is to investigate the effect of a surface treatment with a solution of hydrophobizing agent, spores, tryptic soy broth, calcium lactate and water.

To this end, commercially available concrete cubes from Rocholl GmbH were treated with formulations containing distilled water and optionally WS 405, spores. TSB and/or Ca-Lactat*H₂O. The compositions of the formulations employed in the examples 6a to 6e are reported in table 3a. Example 6e is the reference sample.

TABLE 3a
Formulations employed in example 6

| | Formulation | | | | | Application |
|---|---|---|---|---|---|---|
| | | | | Ca lactate | Dist. | quantity |
| Example | WS405 | Spores | TSB | H2O | water | [g/m²] |
| 6a | 60 g | 0 g | 0 g | 0 g | 90 g | 204 |
| 6b | 60 g | 0 g | 4.5 g | 4.5 g | 90 g | 202.7 |
| 6c | 60 g | 15 g | 4.5 g | 4.5 g | 90 g | 204 |
| 6d | 60 g | 15 g | 4.5 g | 0 g | 90 g | 209.3 |
| 6e | 0 g | 0 g | 0 g | 0 g | 0 g | — |

The cubes were initially immersed in the corresponding formulations until an approximate application quantity of 200 g/m² was achieved. The actual amount of the formulation applied was determined by gravimetric means and is likewise reported in table 3a. After 14 days the reduction in water absorption was determined with the Karsten tube test. To this end, the water absorption was determined after 24 h and related to the water absorption of the reference sample 6e. The results are reported in table 3b.

TABLE 3b
Reduction in water absorption without fracture

| | Water absorption [ml] | | | | Reduction in water |
|---|---|---|---|---|---|
| Product | 0.5 h | 2 h | 6 h | 24 h | absorption [%] |
| 6a | 0 | 0 | 0 | 0.05 | 97.0 |
| 6b | 0 | 0 | 0 | 0.05 | 97.0 |
| 6c | 0 | 0 | 0 | 0.05 | 97.0 |
| 6d | 0 | 0 | 0 | 0.05 | 97.0 |
| 6e | 0.2 | 0.5 | 0.9 | 1.7 | — |

The cubes (including 6e) were then fractured and the fracture surface was brush coated with the respective formulation in the application quantity reported in table 3c. The cubes were then placed on top of one another again at the fracture edges, secured with Teflon tape and then stored in a bowl of water (about 5 mm water fill height) for 14 days so that the crack was immersed in the water on one side. The reduction in water absorption was determined as follows: The cubes were dried and weighed. They were then stored under water for 24 h. From the difference between the masses before and after underwater storage the reduction in water absorption was determined according to the following formula:

Reduction in water absorption %=[(mass after−mass before)/mass before]/[(mass_reference after−mass_reference before)/mass_reference before]*100

The results of reduction in water absorption are shown in the following table 3c.

TABLE 3c
Reduction in water absorption after fracture

| | Application | Reduction in water |
|---|---|---|
| Example | [g/m²] | absorption [%] |
| 6a | 201.3 | 94.9 |
| 6b | 199.3 | 91.6 |
| 6c | 210.7 | 93.2 |
| 6d | 202.7 | 93.8 |
| 6e | — | 4.2* |

*= water absorption after 24 h in %, (mass after − mass before)/mass before *100 = absolute water absorption

As is apparent from table 3c a reduction in water absorption compared to the untreated test specimen is observable even after fracture of the test specimen (cube) and subsequent treatment with the inventive composition.

A Karsten tube test was performed after a storage of 8 weeks. To this end, the tubes were attached above the crack. The side that was stored below the water surface was during the 8 weeks was used. For example 6d no water absorption was observed during a measurement duration of 0.5 h. This means that the crack has closed for this formulation without Ca lactate.

### Example 7: Encapsulation with Polyvinyl Alcohol (PVA)

In this experiment the stability of uncoated and PVA-coated spores of the strain Bacillus subtilis DSM 32315 was analyzed during concrete mixing.

Coating of Bacillus subtilis Spores with Polyvinyl Alcohol:

The apparatus employed for coating/encapsulation was a Hüttlin coater (Bosch) fitted with a fluidized bed attachment. To achieve coating/encapsulation the biomass was initially charged into the Hüttlin coater, sprayed with an aqueous PVA solution and subsequently dried. The biomass employed was a mixture of 50% by weight of Bacillus subtilis DSM 32315 spores and 50% by weight of lime. The PVA solution employed was a solution of 5% by weight of Kuraray Poval® 4-88 PVA and 5% by weight of Kuraray Poval® 8018 PVA in water. The total concentration of PVA was accordingly 10% by weight based on the total mass of the solution. To produce the PVA solution, a mixture of Kuraray Poval® 4-88 PVA and Kuraray Poval® 8018 PVA was initially sprinkled into cold water with stirring and heated to 90° C. to 95° C. in a water bath until fully dissolved before the solution was cooled with stirring to avoid skin formation. Subsequently the biomass was initially charged into the fluidized bed unit, heated with a temperature-controlled nitrogen stream and fluidized. As soon as the fluidized bed had reached the required temperature the PVA solution was added via a peristaltic pump. The relevant process settings are summarized in table 4.

TABLE 4
Settings for fluidized bed process

| Parameter | Unit | Value |
|---|---|---|
| N₂ temperature | ° C. | 60-65 |
| Bed temperature | ° C. | 45-48 |
| N₂ flow rate | m³/h | 20 |
| Spraying air pressure | bar | 0.5 |
| Microclimate | mbar | 150 |
| Pump speed | % | 5 |
| PVA spraying rate | g/h | 92 ± 23 |

In the coating/encapsulation of the biomass 750 g of the aqueous PVA solution (10% by weight PVA) were applied to 500 g of biomass. This corresponds to a proportion of 13% by weight of PVA based on the total mass of the dried product.

Determination of Stability:

To determine stability, equivalent amounts of coated (78 g per 50 l concrete batch, corresponds to 0.5% by weight based on cement) and uncoated spores (46 g per 50 l concrete batch, corresponds to 0.29% by weight based on cement), said amounts being adjusted to the spore concentration CFU/g in the feedstock, were placed in a cement mixer together with the growth medium (92 g of TSB). After 1 min of dry mixing samples were taken before the appropriate amount of water (7.2 kg) was added to the concrete batch. Samples were taken again after a total of 3 min. 20 min. 60 min and 120 min. The samples taken were immediately and in duplicate diluted to about 1:100 in water, shaken and subsequently aliquoted and stored at −20° C. until further processing.

To determine the spore count of the samples the samples were thawed and in a serial dilution diluted in polysorbate peptone salt solution (pH=7) such that after plating-out of the samples and incubation at 37° C. a countable number of colonies on TSA agar plates was to be expected.

TABLE 5
Stability of coated and uncoated DSM
32315 spores during concrete mixing

| Sample | | | Spore count in |
|---|---|---|---|
| formulation | Mixing time | Sample | concrete CFU/g |
| uncoated | — | expected CFU/g | 1.39E+08 |
| | | concrete | |
| PVA-coated | — | expected CFU/g | 1.39E+08 |
| | | concrete | |
| uncoated | 1 min | 1-1 | 5.25E+07 |
| PVA-coated | (dry mixing) | 1-2 | 4.52E+07 |
| | | 1-1 | 6.57E+07 |
| | | 1-2 | 7.01E+07 |
| uncoated | 3 min | 2-1 | 9.53E+07 |
| PVA-coated | (following | 2-2 | 9.84E+07 |
| | addition | 2-1 | 9.28E+07 |
| | of water) | 2-2 | 1.28E+08 |
| uncoated | 20 min | 3-1 | 9.82E+07 |
| PVA-coated | | 3-2 | 6.89E+07 |
| | | 3-1 | 9.08E+07 |
| | | 3-2 | 1.18E+08 |
| uncoated | 60 min | 4-1 | 8.10E+07 |
| PVA-coated | | 4-2 | 7.91E+07 |
| | | 4-1 | 9.01E+07 |
| | | 4-2 | 9.29E+07 |
| uncoated | 120 min | 5-1 | 5.39E+07 |
| PVA-coated | | 5-2 | 5.28E+07 |
| | | 5-1 | 8.91E+07 |
| | | 5-2 | 1.07E+08 |

The results reported in table 5 show that the spores in the concrete batch are probably not yet homogeneously distributed after one minute, thus initially resulting in a lower spore count than expected. After mixing for three minutes the spore count was close to the expected spore count both in the batch comprising coated spores and in the batch comprising uncoated spores. In the course of mixing up to 2 h it was apparent from the spore count data that no loss greater than one log step was incurred either with coated spores or with uncoated spores.

1: A composition comprising at least one microorganism capable of forming a phosphate or carbonate precipitate in an alkaline medium, and optionally at least one calcium source,
wherein the composition comprises at least one silicon compound comprising at least one Si atom, at least one C atom, and at least one H atom.

2: The composition according to claim 1, wherein the at least one microorganism is selected from a bacterium, a lyophilized bacterium, and a bacterial spore of a bacterium.

3: The composition according to claim 1, wherein the at least one microorganism is selected from a bacterial spore or a bacteria of the genera Enterococcus, Diophrobacter, Lysinbacillus, Planococcus, Bacillus, Proteus or Sporosarcina.

4: The composition according to claim 1, wherein a weight ratio of the at least one microorganism capable of forming a phosphate or carbonate precipitate in an alkaline medium to the at least one silicon compound comprising at least one Si atom, at least one C atom, and at least one H atom is from 100:1 to 1:100.

5: The composition according to claim 1, wherein a mass fraction of the at least one microorganism capable of forming a phosphate or carbonate precipitate in an alkaline medium, based on the total mass of the composition, is from 0.0001% to 10% by weight.

6: The composition according to claim 1, wherein the composition contains at least one mineral building material.

7: The composition according to claim 1, wherein the composition contains at least one enrichment medium (growth medium) for enrichment of the at least one microorganism.

8: The composition according to claim 1, wherein the at least one silicon compound comprising at least one Si atom, at least one C atom, and at least one H atom has hydrophobizing properties.

9: The composition according to claim 1, wherein the at least one silicon compound comprising at least one Si atom, at least one C atom, and at least one H atom is selected from silane compounds, siloxane compounds, silicone oils, siliconates, organosilane compounds, or organosiloxane compounds.

10: The composition according to claim 1, wherein the composition contains at least one silicon compound which comprises at least one Si atom, at least one C atom, and at least one H atom, and conforms to a formula (I), (IIa), or (IIb);
wherein formula (I) is represented by R—SiR¹ₓR²_{z},
in which
R is a linear or branched alkyl group having 1 to 20 C atoms,
R¹ is a linear or branched alkyl group having 1 to 4 C atoms,
R² is a linear or branched alkoxy group having 1 to 4 C atoms or a hydroxyl group,
wherein the radicals R¹ and R² may each be identical or different,
x equals 0, 1, or 2,
z equals 1, 2, or 3, and
x+z=3; and
wherein formula (IIa) is represented by (R′)₃Si—O—[Si(R′)₂—O]ₘ—Si(R′)₃
and formula (IIb) is represented by 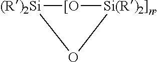
in which the individual radicals R′, independently of one another, represent hydroxyl alkoxy, alkoxyalkoxy, alkyl, alkenyl, cycloalkyl, and/or aryl, and wherein
m is an integer from 2 to 30,
n is an integer from 3 to 30, and
with the proviso that a sufficient number of the individual radicals R′ in the compounds of formulae (IIa) and (IIb) are an alkoxy radical, to ensure that a quotient of the molar ratio of Si to alkoxy radicals in the compounds of formulae (IIa) and (IIb) is at least 0.3.

11: The composition according to claim 1, wherein the at least one silicon compound comprising at least one Si atom, at least one C atom, and at least one H atom is selected from the group consisting of CH₃Si(OCH₃)₃, CH₃Si(OC₂H₅)₃, C₂H₅Si(OC₂H)₃, i-CH₇Si(OC₂H₅)₃, C₂H₅Si(OCH₃)₃, i-C₃H₇Si(OCH₃)₃, n-C₃H₇Si(OCH₃)₃, n-C₃H₇Si(OC₂H), i-C₃H₇Si(OCH)₃, n-C₄H₉Si(OCH₃)₃, n-C₄H₉Si(OC₂H₅)₃, i-C₄H₉Si(OCH₃)₃, n-C₄H₉Si(OC₂H₅)₃, n-C₅H₁₁Si(OCH₃)₃, n-C₅H₁₁Si(OC₂H₅)₃, i-C₅H₁₁Si(OCH₃)₃, i-C₅H₁₁Si(OC₂H₅)₃, n-C₆H₁₃Si(OCH₃)₃, n-C₆H₁₃Si(OC₂H₅)₃, i-C₆H₁₃Si(OCH₃)₃, i-C₆H₁₃Si(OC₂H₅)₃, n-C₈H₁₇Si(OCH₃)₃, n-C₈H₁₇Si(OC₂H)₃, i-C₇H₁₇Si(OCH₃)₃, i-C₈H₁₇Si(OC₂H₅)₃, n-C₁₀H₂₁Si(OCH₃)₃, n-C₁₀H₂₁Si(OC₂H₅)₃, i-C₁₀H₂₁Si(OCH₃)₃, i-C₁₀H₂₁Si(OC₂H₅)₃, n-C₁₆H₃₃Si(OCH₃)₃, n-C₁₆H₃₃Si(OC₂H₅)₃, i-C₁₆H₃₃Si(OCH₃)₃, i-C₁₆H₃₃Si(OC₂H₅)₃, a partial condensate of one or more of the recited compounds, a mixture of the recited compounds, a mixture of the partial condensates, and a mixture of the compounds and the partial condensates.

12: A process for producing a building product, the process comprising:
mixing the composition of claim 1 with a building material.

13: The process according to claim 12, wherein the building product is mortar, a mortar-based component or product, steel-reinforced concrete, concrete, a steel-reinforced concrete part, a concrete part, a concrete block, a roof tile, a brick, or a porous concrete block.

14: The process according to claim 12, wherein the composition is employed before completion of the building product or of a built structure.

15: The process according to claim 12, wherein the composition is employed after completion of the building product or of built structure.

16: The composition according to claim 3, wherein the at least one microorganism is a bacterial spore or a bacteria selected from the group consisting of Bacillus cohnii, Bacillus megaterium, Bacillus pasteurii, Bacillus pseudofirmus, Bacillus sphaericus, Bacillus spp., Bacillus subtilis, Proteus vulgaris, Bacillus licheniformis, Diophrobacter sp., Enterococcus faecalis, Lysinbacillus sphaericus, Proteus vulgaris, and Sporosarcina pasteurii.

17: The composition according to claim 3, wherein the at least one microorganism is a bacterial spore or a bacteria of Bacillus subtilis or Bacillus cohnii.

18: The composition according to claim 3, wherein the at least one microorganism is a bacterial spore or a bacteria of Bacillus subtilis.

19: The composition according to claim 6, wherein the at least one building material is cement.

20: The composition according to claim 7, wherein the at least one enrichment medium is tryptic soy broth.
